(19) United States
(12) Patent Application Publication
(10) Pub. No.: US 2010/0221841 A1
(43) Pub. Date: Sep. 2, 2010

(54) SEMICONDUCTOR DNA SENSING DEVICE AND DNA SENSING METHOD

(76) Inventors: Tetsuya OSAKA, Tokyo (JP); Daisuke Niwa, Tokyo (JP); Norikazu Motohashi, Tokyo (JP)

Correspondence Address: BIRCH STEWART KOLASCH & BIRCH, PO BOX 747, FALLS CHURCH, VA, 22040-0747, US

(21) Appl. No.: 12/717,074

(22) Filed: Mar. 3, 2010

Related U.S. Application Data
(62) Division of application No. 11/514,843, filed on Sep. 5, 2006, now abandoned.

(30) Foreign Application Priority Data
Mar. 3, 2006 (JP) 2006-057706

(51) Int. Cl. G01N 33/50

(52) U.S. Cl. 436/94

(57) ABSTRACT

A semiconductor DNA sensing device is provided herein, which includes a detection section with a field-effect transistor including a semiconductor substrate and a first insulator layer formed thereon as a reactive gate insulator, the first insulating layer including silicon oxide or an inorganic oxide, a first organic monolayer formed on the first insulator layer, the first organic monolayer comprising an organic molecule having a reactive functional group, and a probe DNA containing 3 to 35 nucleotides bonded to the first organic monolayer by the reactive functional group either directly or by an intervening crosslinker, the structure of the probe DNA/the first organic monolayer/the insulating layer/the semiconductor constituting the detection section.

### CROSS-REFERENCE TO RELATED APPLICATION

This application is a Divisional of U.S. patent application Ser. No. 11/514,843, filed Sep. 5, 2006. This application also claims priority under 35 U.S.C. §119(a) on Patent Application No. 2006-057706 filed in Japan on Mar. 3, 2006, the entire contents of which are hereby incorporated by reference.

### TECHNICAL FIELD

This invention relates to a semiconductor DNA sensing device and a DNA sensing method using a field-effect transistor (FET).

### BACKGROUND ART

Biosensing device is widely used in the fields of medicine, environmental studies, and drug discovery. Among such device, a DNA sensing device which can be used in gene therapy and personalized medicine is highly demanded in view of the development in the genome industrial fields.

Mainstream of the DNA sensing has been sensing using fluorescence and luminescence, and more recently, attempts have been made to detect an electrochemical reaction by means of electric current or potential. Examples of the detection using a semiconductor include those based on ion sensitive field-effect transistor (ISFET) having the structure of a silicon nitride layer/a silicon oxide layer/silicon.

However, in these methods, improvements in the DNA detection and assay were accomplished in a quantitative manner, for example, by increasing the effective surface area of the electrode section, increasing the amount of reactants immobilized, or introducing a sensitizing label agent or an intercalator molecule, and improvement of the device itself has been rather rare. On the other hand, the detection using a laser scanner or an electrochemical reaction is associated with the problem that decrease in the response sensitivity (strength and response speed) is likely to be caused by the integration and size reduction.

The semiconductor sensing based on an ISFET is also associated with the difficulty of size reduction and on-chip detection since provision of another glass electrode or the like as the reference electrode is required for the detection. When a pseudo reference electrode is used for the reference electrode, such constitution is also associated with the problem of insufficient accuracy and sensitivity. In addition, the silicon nitride layer used for the sensing membrane of the device is as thick as about 100 to 200 nm, and there is a concern about the decrease in the sensitivity.

As described above, the prior art techniques have been associated with various difficulties for fulfilling the demands of realizing an on-chip device, size reduction, integration, and the like, and a fundamental improvement would be required to maximize the detection efficiency of the DNA sensing, and in particular, detection of SNPs or the like.

### DISCLOSURE OF THE INVENTION

An object of the present invention is to provide a semiconductor DNA sensing device having a detection section comprising the structure of an organic monolayer/an insulating layer/a semiconductor which has the organic monolayer integrally formed with the semiconductor structure, which enables a convenient DNA sensing at a high accuracy. In particular, the object of the present invention is to provide a semiconductor DNA sensing device and a DNA sensing method using a probe DNA containing 3 to 35 nucleotides which are capable of detecting the DNA having a sequence fully complementary to the probe DNA or a DNA comprising a sequence having 1 to 3 base mismatches to the fully complementary DNA.

The inventors of the present invention have made an intensive investigation in order to solve the problems as described above, and found that a semiconductor device having a detector section comprising the structure of a probe DNA/an organic monolayer/an insulator layer/a semiconductor is capable of realizing a convenient and highly accurate detection of an oligo DNA containing 3 to 35 nucleotides by hybridization as well as detection of a mismatch (for example, single nucleotide polymorphism), and that an efficient on-chip DNA sensing is realized by providing a reference section comprising the structure of an organic monolayer/an insulator layer/a semiconductor on the semiconductor device.

A first embodiment of the invention provides a semiconductor DNA sensing device having a detection section comprising an FET comprising a semiconductor substrate and a first insulator layer formed thereon as a reactive gate insulator, the first insulating layer comprising silicon oxide or an inorganic oxide,

a first organic monolayer formed on the first insulator layer, the first organic monolayer comprising an organic molecule having a reactive functional group, and a probe DNA containing 3 to 35 nucleotides bonded to the first organic monolayer by the reactive functional group either directly or by an intervening crosslinker, the structure of the probe DNA/the first organic monolayer/the insulating layer/the semiconductor constituting the detection section.

In a preferred embodiment of the device, the device is constituted such that, when a target DNA which is a DNA having a sequence fully complementary to the probe DNA or a DNA having a sequence having 1 to 3 base mismatches to the fully complementary DNA is reacted with the probe DNA, the target DNA hybridizes with the probe DNA to cause a change in negative charge of the probe DNA, which in turn causes a change in surface potential of the insulating layer which is to be detected.

In another preferred embodiment of the device, the device further comprising a reference section comprising a semiconductor substrate and a second insulator layer formed thereon as a reactive gate insulator, the second insulating layer comprising silicon oxide or an inorganic oxide, and a second organic monolayer formed on the second insulator layer, the second organic monolayer comprising an organic molecule which reacts with neither of the probe DNA and the target DNA, and the structure of the second organic monolayer/the second insulating layer/the semiconductor constituting the reference section.

The first organic monolayer is preferably made of a monolayer of an alkoxysilane having a straight chain hydrocarbon group containing 3 to 20 carbon atoms which has amino functional group, carboxyl functional group, or mercapto functional group. The second organic monolayer is preferably made of a monolayer of an alkoxysilane having a straight chain alkyl group or fluoroalkyl group containing 8 to 22 carbon atoms.

A second embodiment of the invention provides a DNA sensing method comprising the steps of providing a DNA sensing device comprising an FET comprising a semiconductor substrate and a first insulator layer formed thereon as a reactive gate insulator, the first insulating layer comprising silicon oxide or an inorganic oxide; a first organic monolayer formed on the first insulator layer, the second organic monolayer comprising an organic molecule having a reactive functional group; and a probe DNA containing 3 to 35 nucleotides bonded to the first organic monolayer by the reactive functional group either directly or by an intervening crosslinker; and reacting a target DNA which is a DNA having a sequence fully complementary to the probe DNA or a DNA comprising a sequence having 1 to 3 base mismatches to the fully complementary DNA with the probe DNA so that the target DNA hybridizes with the probe DNA to cause a change in negative load of the probe DNA, which in turn causes a change in surface potential of the insulating layer which is to be detected.

In the present invention, the structure of an organic monolayer/an insulator layer/a semiconductor is formed in the gate section of an FET, and the insulating layer comprising silicon oxide or an inorganic oxide is formed to a thickness as thin as an that of an ordinary semiconductor device, and the surface characteristic of this insulating layer is drastically converted by providing an ultra-thin organic monolayer which has a thickness of up to 3 nm. The probe DNA molecule can be arranged on this organic monolayer in an ideal manner either directly or by using an intervening crosslinker. Use of the FET has also enabled a detection without using a label molecule, and such device has superior convenience in use. Furthermore, this structure of the organic monolayer/the insulator layer/the semiconductor can also be applied to the reference device, and the DNA sensing device can be provided as a fully on-chip DNA sensing device.

The semiconductor DNA sensing device of the present invention is extremely effective as an on-chip, high-sensitivity, micro multi-DNA sensing device, and an integrated device produced by using such semiconductor DNA sensing device is capable of sensing a DNA including a mismatch sequence such as single nucleotide polymorphism. Furthermore use of such device together with a reference device enables an on-chip, convenient, high-sensitivity DNA sensing which is indispensable for an advanced medicine and personalized medicine.

### BRIEF DESCRIPTION OF THE DRAWINGS

FIG. 1 is a cross-sectional view of the semiconductor DNA sensing device of the present invention. FIG. 1A shows an FET, FIG. 1B shows the FET having an organic monolayer formed on the insulating layer of the gate electrode, and FIG. 1C shows the state having the probe DNA immobilized on the organic monolayer by an intervening molecule.

FIG. 2 is a schematic view illustrating the DNA detection by means of hybridization using the semiconductor DNA sensing device of the present invention.

FIG. 3 shows an on-chip device unit according to an embodiment of the present invention. FIG. 3A is a plan view, and FIG. 3B is an exploded cross-sectional view.

FIG. 4 shows the substrate used in Experimental Example 1 having an amino monolayer on which the probe DNA can be immobilized and a fluoroalkyl monolayer which does not react with the DNA formed in a particular pattern. FIG. 4A is a plan view, and FIG. 4B is an exploded cross-sectional view.

FIG. 5 shows photographs taken by a fluorescence microscope after immobilizing the probe DNA in Experimental Example 1. FIG. 5B is a partially exploded photograph of FIG. 5A.

FIG. 6 is a graph showing current-voltage curves before and after the hybridization in Example 1.

### DESCRIPTION OF THE PREFERRED EMBODIMENTS

The semiconductor DNA sensing device of the present invention has a detection section, and this detection section comprises the structure of a probe DNA/a first organic monolayer/an insulating layer/a semiconductor. More specifically, the DNA sensing device comprises an FET comprising a semiconductor substrate and a first insulator layer formed thereon, the first insulating layer comprising silicon oxide or an inorganic oxide and serving as a reactive gate insulator; a first organic monolayer formed on the first insulator layer, the first organic monolayer comprising an organic molecule having a reactive functional group; and a probe DNA containing 3 to 35 nucleotides bonded to the first organic monolayer by the reactive functional group either directly or by an intervening crosslinker.

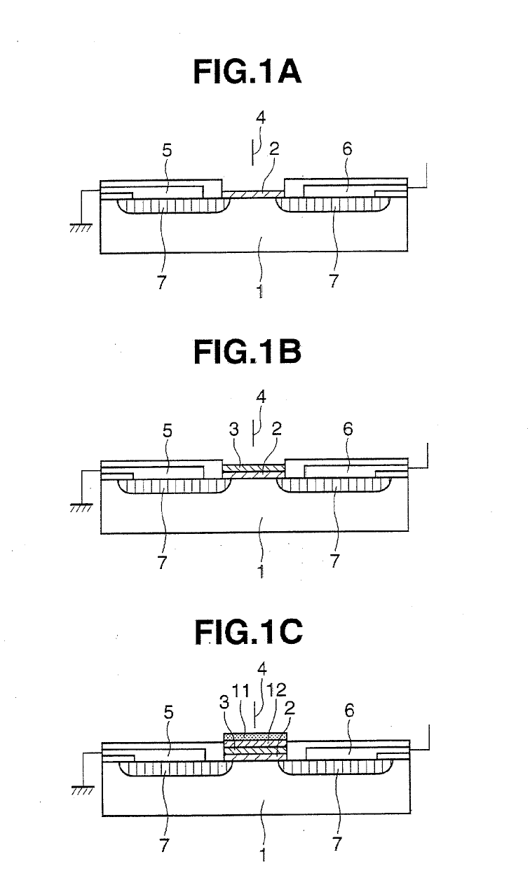

As described above, the detection section in the semiconductor DNA sensing device of the present invention has the structure of a probe DNA/a first organic monolayer/an insulating layer/a semiconductor, (and when the probe DNA is bonded by an intervening crosslinker, the structure of a probe DNA having the intervening crosslinker bonded thereto/a first organic monolayer/an insulating layer/a semiconductor). Of such structure, the structural part of the insulating layer/the semiconductor may comprise an FET of known constitution comprising a semiconductor substrate and an insulator layer formed thereon, and the insulating layer may comprise silicon oxide or an inorganic oxide to serve as a reactive gate insulator. An exemplary FET is the one shown in FIG. 1A. In FIG. 1, a silicon substrate 1 has an insulator layer 2 comprising silicon oxide or an inorganic oxide (for example, glass or alumina), a gate electrode 4, a source electrode 5, and a drain electrode 6, and channel region 7.

As shown in FIG. 1B, the organic monolayer 3 is formed on the insulator layer 2. With regard to the basic principle of the detection of the present invention, it is the change in the surface potential associated with the hybridization of the DNA on the insulator layer that is detected in terms of electric signal. The insulator layer can be formed to a thickness of 10 to 100 nm, and in particular, to a thickness of 10 to 50 nm.

The organic monolayer is formed directly on the insulator layer. This organic monolayer is formed on the insulator layer by gas-phase or liquid-phase reaction of the organic molecule, and by optimizing the reaction, for example, by utilizing self assembly function of the monomolecules, to thereby produce a layer having the organic monomolecules closely packed.

In this case, the organic monolayer preferably comprises an alkoxysilane having a straight chain hydrocarbon group (for example, an alkyl group) containing 3 to 20 carbon atoms having at least one reactive functional group, in particular, an amino functional group (for example, —NH₂, —NH—, C₅H₅N—, and C₄H₄N—), a carboxyl functional group (for example, —COOH), or a mercapto functional group (for example, —SH). Use of such alkoxysilane is preferable since, when the insulator layer is formed from a silicon oxide, it can be directly bonded to the silicon oxide of the insulator layer.

Alternatively, a monolayer may be formed from an alkoxysilane having an amino derivatizing group such as —Br or —CN which can be substituted with the reactive functional group such as the amino functional group, the carboxyl functional group, or the mercapto functional group, and then, the amino group may be introduced by substitution of the amino derivatizing group with the amino group.

When the insulator layer comprises a silicon oxide, the alkoxysilane used is preferably a trialkoxysilane in view of the adhesion, and the alkoxy group is preferably an alkoxy group containing 1 to 3 carbon atoms (—OR wherein R represents a monovalent hydrocarbon group), and in particular, methoxy group (—OCH₃) or ethoxy group (—OC₂H₅). Exemplary such alkoxysilanes include trialkoxysilanes having a reactive functional group such as H₂N(CH₂)₃Si (OC₂H₅)₃.

In the semiconductor DNA sensing device of the present invention, a probe DNA containing 3 to 35 nucleotides (a oligonucleotide) is bonded to the organic monolayer either directly or by an intervening crosslinker, for example, as shown in FIG. 1C in which a probe DNA 11 containing 3 to 35 nucleotides is bonded to the organic monolayer by an intervening crosslinker 12.

When the functional group of the probe DNA and the reactive functional group of the organic molecule constituting the organic monolayer are mutually reactive and capable of forming a bond, the probe DNA can be immobilized by direct reaction between such functional groups. On the other hand, when the functional group of the probe DNA and the reactive functional group of the organic molecule constituting the organic monolayer are not mutually reactive and incapable of forming a bond therebetween, the probe DNA may be bonded to the organic molecule constituting the organic monolayer by an intervening crosslinker. In such a case, for example, when the monolayer is the one comprising an organic molecule having amino group as the reactive functional group, an organic molecule such as the one having aldehyde group on opposite ends of glutaraldehyde or the like may be used so that the aldehyde group on one end reacts with the organic monolayer and the aldehyde group on the other end reacts with the amino group of the probe DNA to thereby immobilize the probe DNA. The probe DNA use may be either the one solely comprising the nucleotide chain or the one modified with amino group or mercapto group.

Introduction of the crosslinker can be accomplished by immersing the FET in a solution containing the crosslinker, and bringing the crosslinker in contact with the organic monolayer formed on the insulator layer. On the other hand, introduction of the probe DNA can be accomplished by immersing the FET in a solution containing the probe DNA, if desired, after adding a crosslinker in the solution, to bring the probe DNA in contact with the organic monolayer formed on the insulator layer or the crosslinker.

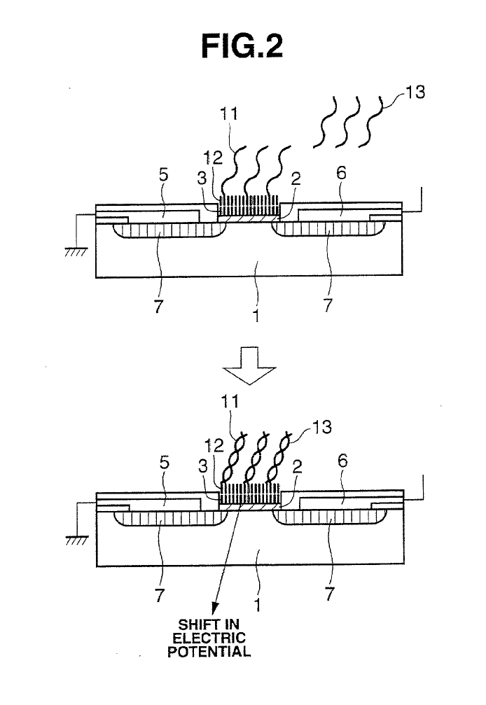

FIG. 2 is a schematic view showing the DNA detection based on hybridization using the semiconductor DNA sensing device of the present invention. In this DNA sensing, a DNA having an equivalent length with the probe DNA which has either the sequence fully complementary with the probe DNA or a sequence with 1 to 3 base mismatches with the probe DNA is reacted as a target DNA with the probe DNA immobilized on the organic monolayer by means of an intervening crosslinker, and the change in the surface potential of the insulator layer associated with the change in the negative charge of the probe DNA caused by the hybridization is detected in terms of electric signal. In FIG. 2, reference numeral 13 designates the target DNA, and explanation of the constitution of the device is omitted by using the same reference numerals as those used in FIG. 1.

When a DNA fully complementary to the probe DNA is reacted with the probe DNA, they easily form a double helix, and the surface potential of the gate electrode shifts to the negative side by the hybridization since a DNA is negatively charged due to the phosphate group. When a p-FET is used in such a case, the threshold voltage shifts to the positive side, and the signal detected will be the shift in the potential when a constant electric current is applied whereas the signal detected will be the shift in the electric current when a constant voltage is applied. When an n-FET is used, the threshold voltage will shift to the positive side which is opposite to the case using the p-FET.

In the meanwhile, when a molecule having a base mismatch is used for the target DNA, progress and degree of the double helix formation and the double helix structure formed will be different from those of the target DNA fully complementary to the probe DNA, and the threshold voltage will shift to a positive or negative side. The base mismatch in the DNA will then be detected by using such sift.

In the present invention, a second insulator layer comprising silicon oxide or an inorganic oxide may be formed on the semiconductor of the FET as a reference gate insulator. By forming a second organic monolayer comprising an organic molecule which reacts with neither of the probe DNA and the target DNA, the structure of the organic monolayer/the insulator layer/the semiconductor can be used as a reference section. When the reactive gate insulator and the reference gate insulator are arranged at a distance sufficient to prevent mutual influence in measuring the shift in the potential, the first insulator layer of the reactive gate insulator and the second insulator layer of the reference gate insulator can be formed in the same layer.

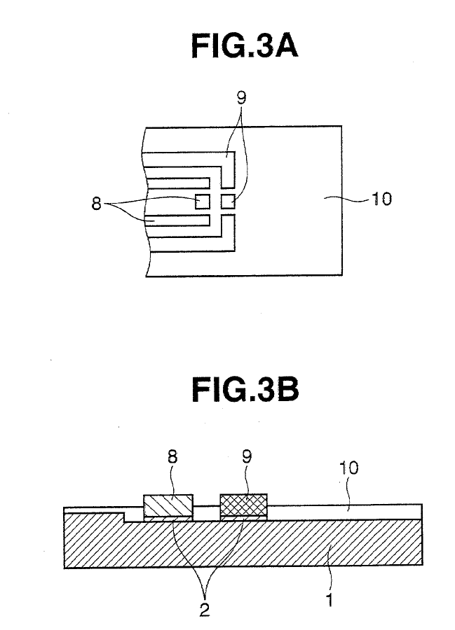

FIG. 3 shows an exemplary unit constitution of the on chip device wherein the structure of the organic monolayer/the insulator layer/the semiconductor is applied for the detection section 9 and the reference section 8. In FIG. 3, a silicon substrate 1 has an insulator layer 2 and a temperate section 10. The constitution of this device is not limited to the one shown in FIG. 3, and the detector section and the reference section does not necessarily be arranged in pair, and the detector section and the reference section of various number may be arranged in various combinations. The detector section and the reference section may be respectively formed to a size of several μm to several tens μm.

A second organic monolayer comprising an organic molecule which reacts with neither of the probe DNA and the target DNA is formed on the second insulator layer of the reference section, and preferably, this organic monolayer comprises a monolayer of an alkoxysilane having a straight chain alkyl group or fluoroalkyl group containing 8 to 22 carbon atoms.

The organic monolayer is preferably a self-assembled layer in view of forming a uniform film on the insulator layer. Exemplary layers include those comprising an alkylsilane such as CH₃ (CH₂)₁₇Si(OCH₃)₃ and a fluoroalkylsilane such as CF₃ (CF₂)₇ (CH₂)₂Si(OCH₃)₃. When the organic monolayer is formed from an alkoxysilane, the insulator layer is preferably the one comprising silicon oxide.

The first and the second organic monolayers can be formed at any desired position by patterning. The patterning of the organic monolayer is particularly effective for producing an on-chip integrated device. For example, a first organic monolayer comprising an organic molecule having a reactive functional group is formed on the surface of the insulator layer of the detector section to enable the DNA immobilization, while a second organic monolayer comprising an organic molecule which reacts with neither of the probe DNA and the target DNA is formed in a position-selective manner in the reference section, and in the non-gate area (i.e. temperate section), in order to avoid the non-specific adsorption of the DNA.

The patterning can be accomplished by forming an organic monolayer comprising an organic molecule having no reactivity with the probe DNA or the target DNA on the entire surface of the insulator layer that had been formed on the substrate so that the organic monolayer serves a template; coating a resist for a particle beam (or UV, electron beam, X ray, etc.); conducting the patterning by removing the part of the resist above the detector section with the particle beam (or UV, electron beam, X ray, etc.); removing the organic monolayer that had become exposed in the opening of the resist pattern by means of oxygen plasma etching or the like; and thereafter forming an organic monolayer of an organic molecule having a reactive functional group on the detector section.

The immobilization of the probe DNA is preferably accomplished by using a probe DNA dissolved in a buffer which is preferably neutral to acidic. When the probe DNA is immobilized by an intervening crosslinker, the immobilization is preferably conducted by reacting the crosslinker with the organic monolayer, and thereafter removing the non-specifically binding probe DNA by washing with a buffer.

When the target DNA is hybridized after immobilizing the probe DNA, the target DNA is preferably used by dissolving in a buffer which is preferably equivalent to the one used in the immobilization of the probe DNA. In the assay, use of a buffer equivalent to the one used in the immobilization of the probe DNA is preferable. When two or more types of probe DNAs are immobilized, or when the target DNA is separately reacted with a device having two or more detection sections, spotting and other techniques may be utilized as desired.

### EXAMPLE

The present invention is described in further detail by referring to the following Experimental Examples and Examples which by no means limit the scope of the present invention.

The device used in the Example is the semiconductor DNA sensing device having a detection section comprising the structure of a probe DNA/a first organic monolayer/an insulating layer/a semiconductor and a reference section comprising the structure of a second organic monolayer/a second insulating layer/a semiconductor. The insulator layer used was the one comprising silicon oxide, and the first organic monolayer of the detection section was the one comprising an amino monolayer formed from H₂N(CH₂)₃Si(OC₂H₅)₃, and the second organic monolayer of the reference section was the one comprising a fluoroalkyl monolayer formed from CF₃(CF₂)₇(CH₂)₂Si(OCH₃)₃. The fluoroalkyl monolayer formed from the CF₃(CF₂)₇(CH₂)₂Si(OCH₃)₃ was also used for the part (template section) other than the detection section and the reference section (gate electrode).

### Experimental Example 1

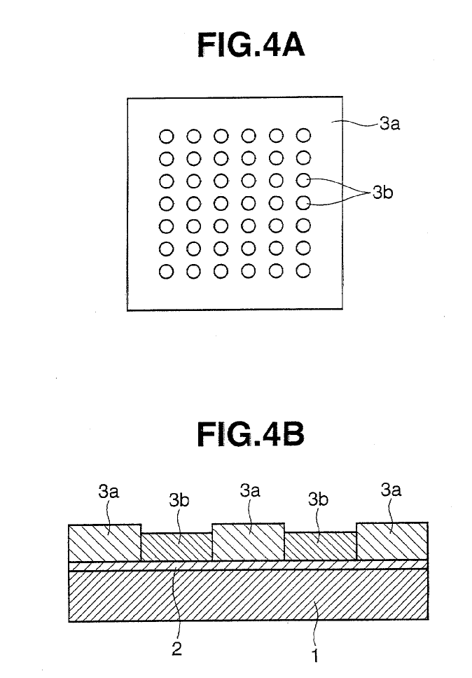

A substrate shown in FIG. 4 having a pattern of an amino monolayer 3b which is adapted for immobilization of the probe DNA and a fluoroalkyl monolayer 3a which does not react with the DNA was prepared, and the substrate was examined to confirm that the DNA had been immobilize in a position-selective manner. In FIG. 4, the substrate comprises a silicon substrate 1 and an insulator layer 2.

The probe DNA used was the one comprising 20 nucleotides which had been modified with thiol 5′-SH-TTTTTTTTTTTTTTTTTTTT-3′) (SEQ ID NO: 1). Sulfo-LC-SPDP was used for the crosslinker between the surface of the amino monolayer to be modified and the probe DNA.

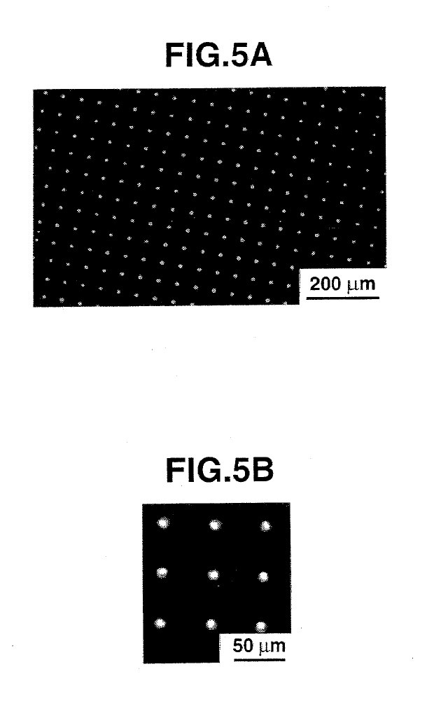

The surface after the probe DNA immobilization was observed using a fluorescence microscope, and it was then revealed that the probe DNA had been immobilized in accordance with the pattern of the amino monolayer formation as shown FIG. 5. In particular, fluorescence intensity of the part where the fluoroalkyl monolayer was present was consistent with the background value of the substrate, and absence of the non-specific adsorption of the DNA to the fluoroalkyl monolayer was thereby demonstrated.

### Example 1

Based on the preliminary results of the Experimental Example 1, hybridization of the DNA having a fully complementary sequence was detected by using the device produced as described above.

The probe DNA was immobilized on the gate electrode of the detection section modified with the amino monolayer. First, the amino molecule was reacted with the glutaraldehyde having aldehyde group on opposite ends for crosslinking of the probe DNA. Next, the probe DNA was immobilized by the reaction in phosphate buffer containing an amino-modified probe DNA containing 20 nucleotides (3′—NH₂-TTTTTTTTTTTTTTTTTTTT-5′) (SEQ ID NO: 2). After washing the substrate, the device having the immobilized probe DNA was evaluated for its current-voltage curve in the phosphate buffer.

Subsequently, hybridization was conducted in phosphate buffer containing a target DNA comprising 20 complementary nucleotides (A20: 5′-AAAAAAAAAAAAAAAAAAAA-3′) (SEQ ID NO: 3). After washing the substrate, current-voltage curve after the hybridization was measured in phosphate buffer to thereby evaluate difference in the voltage response before and after the hybridization.

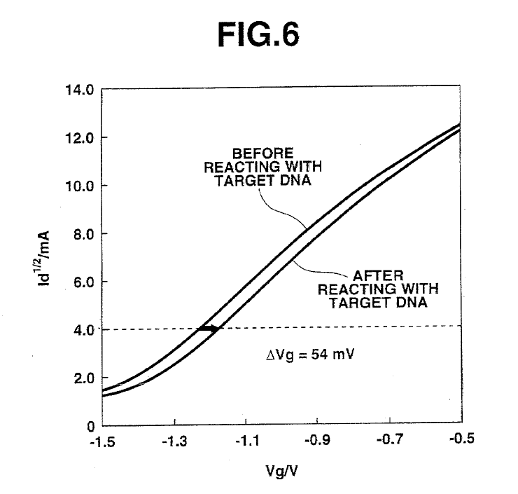

FIG. 6 shows the current-voltage curves before and after the hybridization. As shown in FIG. 6, the response curves shifted in the positive direction. Since the FET used in this Example was a p-FET, this result indicate that the gate surface potential had shifted to the negative side, and this result has adequacy. The shift was as quite substantial at a level of about 50 mV.

On the other hand, when the measurement was carried out by using a non-complementary target DNA (T20: 5′-TTTTTTTTTTTTTTTTTTTT-3′) (SEQ ID NO: 4), only negligible shift in the electric potential (at the level of about 1 mV) was noted before and after the hybridization.

As demonstrated in the results as described above, the device of the present invention is capable of accomplishing the sensing of an oligo DNA fragment.

In the meanwhile, no shift in the voltage was noted in neither of before the immobilization of the probe DNA, after the immobilization of the probe DNA, and after the hybridization of the target DNA in the reference section where the non-reactive fluoroalkyl monolayer had been formed. These results indicated that the structure of the organic monolayer/the insulator layer/the semiconductor having the non-reactive fluoroalkyl monolayer formed would be a functional reference electrode in the DNA sensing.

### Example 2

Hybridization of a fully complementary sequence comprising different nucleotides was detected by using the device as described above. The target DNA used was a DNA modified with amino group at 3′ end, namely, 3′—NH₂-ACGAACATAGCCCGCCTTAC-5′ (SEQ ID NO: 5) and the probe DNA was a fully complementary 5′-TGCTGTTATCGGGCGGAATG-3′ (SEQ ID NO: 6). The voltage response was measured by repeating the procedure of Example 1, and the voltage shift of about 50 mV to the positive side was measured in the DNA comprising the mixed nucleotides. The result indicated that the sensing by the device of the present invention is also realized for the actual DNA comprising the mixed nucleotides.

### Example 3

Mismatch sequence was detected by using a target DNA comprising a mismatch sequence. The probe DNA used was the same as the one used in Example 2, and the target DNAs used were those with single nucleotide polymorphisms having a mismatch of 1, 3, or 5 bases. The DNA with single base mismatch was 5′-TGCTTGTATCGTGCGGAATG-3′ (SEQ ID NO: 7), the DNA with 3 base mismatches was 5′-TGCTAGTATCGTGCGGAGTG-3′ (SEQ ID NO: 8), and the DNA with 5 base mismatches was 5′-AGCTAGTATCGTGCCGAGTG-3′ (SEQ ID NO: 9).

The procedure of Example 1 was repeated to measure the voltage response. In the case of the DNA comprising 20 nucleotides, a DNA with 5 base mismatches is said to undergo substantially no hybridization, and in view of this, the shift in the voltage in the order of about several mV was an adequate result. In the meanwhile, a voltage shift of about 20 mV was confirmed for the DNA with single base mismatch, and a voltage shift of about 8 mV was confirmed for the DNA with 3 base mismatches.

When the result of the DNA with single base mismatch and the result of the fully complementary DNA (Example 2) are compared, difference in the voltage shift was as much as about 30 mV, this sensitivity is incomparable to other detection method. More specifically, use of the device of the present invention has enabled detection using no label or tag, and this is a significant difference from the conventional detection in which the detection of the fluorescence or electrochemical detection was accomplished by introducing an intercalator molecule or a reactive enzyme or by means of signal amplification using triple strand reaction.

1. A DNA sensing method comprising the steps of
providing a DNA sensing device comprising a field-effect transistor comprising a semiconductor substrate and a first insulator layer formed thereon as a reactive gate insulator, the first insulating layer comprising silicon oxide or an inorganic oxide; a first organic monolayer formed on the first insulator layer, the first organic monolayer comprising an organic molecule having a reactive functional group; and a probe DNA containing 3 to 35 nucleotides bonded to the first organic monolayer by the reactive functional group either directly or by an intervening crosslinker;
reacting a target DNA which is a DNA comprising a sequence having 1 to 3 base mismatches to the fully complementary DNA with the probe DNA so that the target DNA hybridizes with the probe DNA to cause a change in negative charge of the probe DNA, which in turn causes a change in surface potential of the insulating layer which is to be detected; and
detecting the change in surface potential by the field-effect transistor.

2. The DNA sensing method of claim 1, wherein the first organic monolayer is a monolayer of an alkoxysilane having a straight chain hydrocarbon group containing 3 to 20 carbon atoms which has amino functional group, carboxyl functional group, or mercapto functional group.

3. The DNA sensing method of claim 1, wherein the organic monolayer is formed on the insulator layer by gas-phase or liquid-phase reaction of the organic molecule.

4. The DNA sensing method of claim 1, wherein the alkoxysilane is trialkoxysilane containing the alkoxy group containing 1 to 3 carbon atoms.

5. The DNA sensing method of claim 1, wherein the organic monolayer has a thickness of up to 3 nm.

6. The DNA sensing method of claim 1, wherein the probe DNA containing 3 to 35 nucleotides is directly bonded to the first organic monolayer by the reactive functional group.

7. The DNA sensing method of claim 1, wherein the target DNA is a DNA comprising a sequence having 2 or 3 base mismatches to the fully complementary DNA with the probe DNA.


SEQUENCE LISTING (9 sequences)

SEQ ID NO: 1 - DNA, 20 bp, Artificial Sequence
  Synthetic oligonucleotide.  5′- end is terminated by SH group.
tttttttttttttttttttt

SEQ ID NO: 2 - DNA, 20 bp, Artificial Sequence
  Synthetic oligonucleotide.  3′- end is terminated by NH2 group.
tttttttttttttttttttt

SEQ ID NO: 3 - DNA, 20 bp, Artificial Sequence
  Synthetic oligonucleotide.
aaaaaaaaaaaaaaaaaaaa

SEQ ID NO: 4 - DNA, 20 bp, Artificial Sequence
  Synthetic oligonucleotide.
tttttttttttttttttttt

SEQ ID NO: 5 - DNA, 20 bp, Artificial Sequence
  Synthetic oligonucleotide.  3′- end is terminated by NH2 group.
cattccgcccgatacaagca

SEQ ID NO: 6 - DNA, 20 bp, Artificial Sequence
  Synthetic oligonucleotide.
tgctgttatcgggcggaatg

SEQ ID NO: 7 - DNA, 20 bp, Artificial Sequence
  Synthetic oligonucleotide.
tgcttgtatcgtgcggaatg

SEQ ID NO: 8 - DNA, 20 bp, Artificial Sequence
  Synthetic oligonucleotide.
tgctagtatcgtgcggagtg

SEQ ID NO: 9 - DNA, 20 bp, Artificial Sequence
  Synthetic oligonucleotide.
agctagtatcgtgccgagtg